(19) United States
(12) Patent Application Publication
(10) Pub. No.: US 2023/0191102 A1
(43) Pub. Date: Jun. 22, 2023

(54) Antimicrobial Skin Isolator for Catheters

(71) Applicant: Bard Access Systems, Inc., Salt Lake City, UT (US)

(72) Inventors: Bryan Haymond, Emigration Canyon, UT (US); Glade H. Howell, Draper, UT (US)

(21) Appl. No.: 18/082,519

(22) Filed: Dec. 15, 2022

Related U.S. Application Data
(60) Provisional application No. 63/290,565, filed on Dec. 16, 2021.

(51) Int. Cl. A61M 39/02; A61M 25/06; A61M 39/20

(52) U.S. Cl. CPC A61M 39/0247; A61M 25/0662; A61M 39/20; A61M 2025/0681; A61M 2039/0258; A61M 2039/0273; A61M 2039/0285; A61M 2205/0205

(57) ABSTRACT

An antimicrobial medical device for use during the placement of a catheter. The antimicrobial medical device includes a tubular body configured to receive the catheter through a lumen of the tubular body during an intravascular procedure and wall of the tubular body forms a liner between catheter and the patient's skin. The device further includes an antimicrobial material coupled with the tubular body so that in use the antimicrobial material inhibits microbe growth at the insertion site.

### PRIORITY

This application claims the benefit of priority to U.S. Provisional Application No. 63/290,565, filed Dec. 16, 2021, which is incorporated by reference in its entirety into this application.

### BACKGROUND

Intravascular procedures provide for a wide array of the patient treatments. Access to internal organs or portions of the body via vasculature pathways have significant advantages over access via surgery. Despite the advantages, accessing the vasculature introduces risk to the patient, such as exposure to microbial contamination leading to infection or disease transmission. Microbial ingress via a vasculature access pathway is a constant concern for medical practitioners due to the high number of intravascular procedures and consequences of microbial ingress may result in extended patient stays and medical care costs of needed corrective action. Long term catheter procedures exacerbate the occurrence rate and risks associate with microbial ingress. As such, systems and methods for reducing the occurrence rate and severity of the microbial ingress, such as those described herein may provide a significant benefit to patients and healthcare providers, as well as reduce the cost of performing intravascular procedures.

Disclosed herein are systems and methods for providing a barrier for microbial ingress during are intravascular procedures.

### SUMMARY

Disclosed herein is an antimicrobial medical device for use during the placement of a catheter. According to some embodiments, the antimicrobial medical device includes a tubular body that defines a lumen extending between a proximal end and a distal end of the tubular body, where the lumen is configured to receive the catheter therethrough during an intravascular procedure. The device further includes an antimicrobial material coupled with the tubular body, where the antimicrobial material is a coating of the tubular body and/or impregnated into the material of the tubular body. The tubular body is configured for insertion within a vascular access pathway extending through a skin layer of a patient so that, in use, a wall of the tubular body defines a microbial barrier between the skin layer and the catheter.

The device may include a flange protruding radially away from the tubular body, where the flange is configured to extend along a surface of the skin when the tubular body is inserted into the vascular access pathway. The flange may also be configured for attachment to the surface of the skin. In some embodiments, the flange includes an adhesive applied to an underside of the flange. In some embodiments, the flange is oriented at an angle with respect to a longitudinal axis of the tubular body.

The tubular body may be configured to form a seal with the catheter to prevent egress of body fluid between the tubular body and the catheter, and in some embodiments, the tubular body includes an inward annular protrusion configured to form the seal. In some embodiments, the tubular body is sized so that, in use, the distal end of the tubular body is disposed within a blood vessel.

Also disclosed herein is an intravascular catheter assembly, that includes a catheter and the antimicrobial medical device of any of the embodiments described above, where the antimicrobial medical device is coupled with the catheter so that the antimicrobial medical device annularly covers at least a portion of the catheter. In some embodiments, the antimicrobial medical device covers a distal tip of the catheter.

Further disclosed herein is a catheter introducer assembly, that includes a catheter introducer and the antimicrobial medical device of any of the embodiments described above where the antimicrobial medical device is coupled with the catheter introducer so that the antimicrobial medical device annularly covers at least a portion of the catheter introducer. In some embodiments, the antimicrobial medical device covers a distal tip of the catheter introducer.

Also disclosed herein is a method of placing a catheter within a patient. According to some embodiments, the method includes providing an isolator that includes a tubular body defining a lumen extending between a proximal end and a distal end, where the lumen is configured to receive the catheter. The isolator further includes an antimicrobial material coupled with the tubular body. The method further includes: (i) inserting the isolator into a vascular access pathway of the patient, (ii) inserting the catheter through the lumen of the isolator, and (iii) advancing the catheter along a blood vessel of the patient.

In some embodiments, inserting the catheter through the lumen of the isolator forms a fluid seal between the catheter and the isolator and in further embodiments, the isolator defines a microbial barrier between the skin layer of the patient and the catheter.

In some embodiments, the method further includes inserting the isolator into the vascular access pathway so that the distal end of the tubular body is disposed within the blood vessel.

In some embodiments, the method further includes inserting the catheter through the lumen of the isolator after inserting the isolator into the vascular access pathway.

In some embodiments, the method further includes inserting the isolator into the vascular access pathway so that a flange of the isolator is disposed adjacent a skin surface of the patient and/or attaching the flange of the isolator to the skin surface. The method may also include attaching the isolator to the catheter.

In some embodiments, the method further includes inserting the catheter into the lumen of the isolator so that a distal end of the catheter is disposed proximal the distal end of the tubular body prior to inserting the isolator into the vascular access pathway, and the method may further include manually applying a distally oriented force to the catheter, where the distally oriented force is transferred to the isolator via the catheter.

In some embodiments, the method further includes inserting a catheter introducer into the isolator and the method may further include inserting the catheter introducer into the vascular access pathway after inserting the catheter introducer into the isolator.

These and other features of the concepts provided herein will become more apparent to those of skill in the art in view of the accompanying drawings and following description, which describe particular embodiments of such concepts in greater detail.

### DRAWINGS

FIG. 1A illustrates an antimicrobial medical device in use with a catheter in accordance with some embodiments.

FIG. 1B is a cross-sectional side view of the antimicrobial medical device if FIG. 1A in accordance with some embodiments.

FIG. 2A illustrates a catheter assembly including the antimicrobial medical device of FIGS. 1A-1B in accordance with some embodiments.

FIG. 2B is a detailed view of a distal tip of the catheter assembly of FIG. 2A in accordance with some embodiments.

FIG. 3A illustrates a catheter introducer assembly including the antimicrobial medical device of FIGS. 1A-1B in accordance with some embodiments.

FIG. 3B is a detailed view of a distal tip of the catheter introducer assembly of FIG. 3A in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled to each other through an intermediate component. Further, two components may be coupled with each other when one component is integrated into the other component.

With respect to “proximal,” a “proximal portion” or “proximal section” of, for example, an isolator disclosed herein includes a portion or section of the isolator intended to be near a clinician or directed toward the clinician (e.g., away from the patient) when the isolator is used on a patient. A “proximal end” of, for example, the isolator includes an end of the isolator intended to extend away from the skin surface when the isolator is used on the patient. The proximal portion, the proximal section, or the proximal length of the isolator can include the proximal end of the isolator; however, the proximal portion, the proximal section, or the proximal length of the isolator need not include the proximal end of the isolator. That is, unless context suggests otherwise, the proximal portion, the proximal section, or the proximal length of the isolator is not a terminal portion or terminal length of the isolator.

With respect to “distal,” a “distal portion” or a “distal section” of, for example, an isolator includes a portion or section of the isolator intended to be in a patient when the isolator is used on the patient. Likewise, a “distal length” of, for example, the isolator includes a length of the isolator intended to extend into the patient when the isolator is used on the patient. A “distal end” of, for example, the isolator includes an end of the isolator intended to be in the patient when the isolator is used on the patient. The distal portion, the distal section, or the distal length of the isolator can include the distal end of the isolator; however, the distal portion, the distal section, or the distal length of the isolator need not include the distal end of the isolator. That is, unless context suggests otherwise, the distal portion, the distal section, or the distal length of the isolator is not a terminal portion or terminal length of the isolator.

Unless defined otherwise, all technical and scientific terms used herein have the same meaning as commonly understood by those of ordinary skill in the art.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

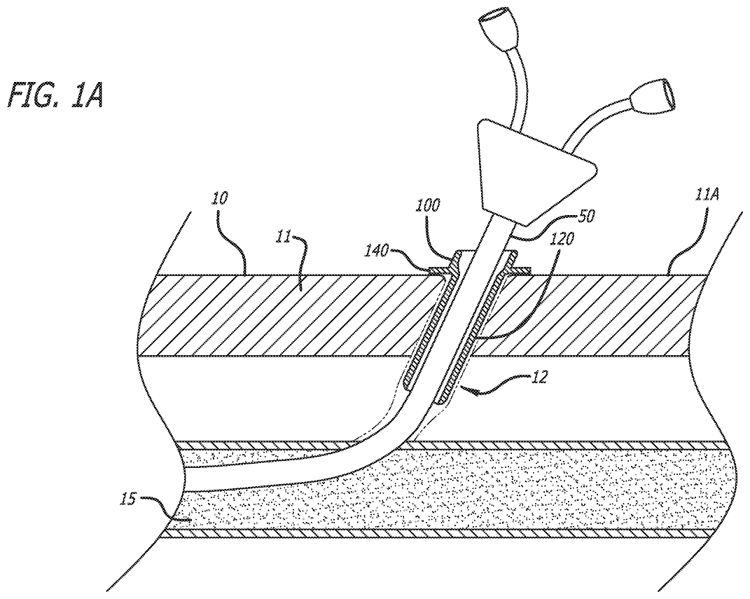

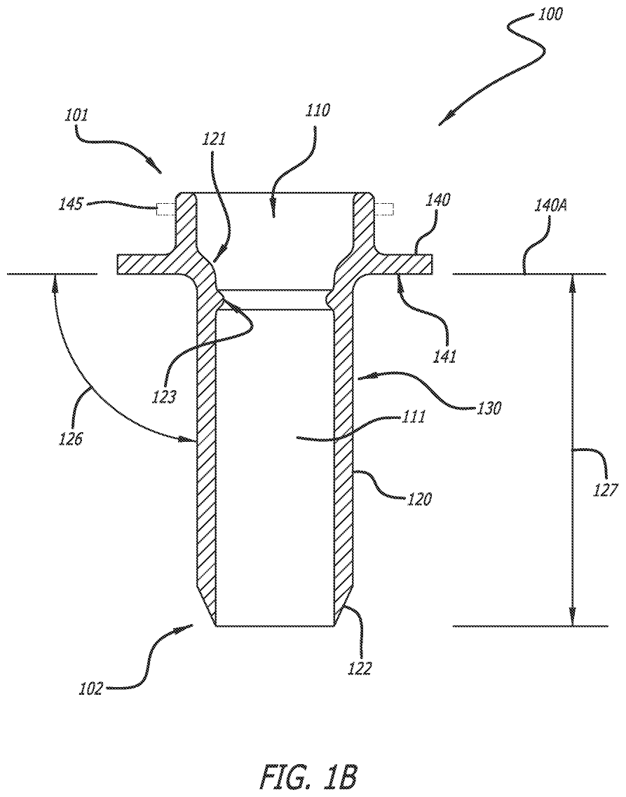

FIGS. 1A-1B illustrate an embodiment of an antimicrobial medical device in the form of an isolator for use with a catheter. The isolator 100 is generally configured to inhibit microbial ingression into a patient during an intravascular procedure. More specifically the isolator 100 defines a microbial barrier extending along at least a portion of the vascular access pathway 12, such as through the skin layer 11 of the patient 10. Preventing patient infection due to microbial ingress at the vascular access site during an intravascular procedure avoids significant patient risk, extended patient stays, and medical care cost. The skin layer 11 of the patient 10 generally forms a microbial barrier for patient 10. As such, an opening through the skin layer 11 such as the pathway 12 provides a pathway for microbes to enter the patient 10. The isolator 100, having antimicrobial properties, is configured to inhibit microbial ingress during and intravascular procedure.

With reference to FIG. 1A, a catheter 50 is shown inserted into the pathway 12 and further into the blood vessel 15. In the illustrated instance of FIG. 1A, the isolator 100 is shown extending through the skin layer 11 (i.e., along only a portion of the pathway 12). However, in other instances, the isolator 100 may extend along an entirety of the pathway 12 from the skin surface 11A to the blood vessel 15. As such, the isolator 100 provides a tubular liner for the catheter 50 extending along at least a skin layer portion of the pathway 12. Hence, the catheter 50 is physically separated from the patient 10 along at least the skin layer portion of the pathway 12 by the tubular wall 120 of isolator 100. The isolator 100 includes antimicrobial properties to kill or inhibit the growth of microbes adjacent the isolator 100 as further discussed below.

In some embodiments, the catheter 50 may be a central line catheter, such as a central venous catheter (CVC), a peripherally inserted central catheter (PICC), or the like. In other embodiments, the catheter 50 may be an intravascular device such as a stylet, a guidewire, an introducer, a dilator, or any other device configured for insertion into or advancement along the vasculature of the patient 10.

In use, the isolator 100 may be inserted along the pathway 12 prior to insertion of the catheter 50. As shown, the catheter 50 is inserted through a lumen of the isolator 100. In some instances, the catheter 50 may remain inserted into the patient 10 for an extended period of the time, e.g., several days or more. As such, the isolator 100 may remain in place within the pathway 12 during the duration of an intravascular procedure. In some instances, the isolator 100 may be removed from the patient 10 at the same time the catheter 50 is removed. In other instances, the isolator 100 may remain in place so that the isolator 100 may be used with a subsequent catheter or other intravascular device.

The isolator 100 may be configured to form fluid seal with the catheter 50 to prevent body fluid (e.g., blood) from migrating along an annular space between the isolator wall 120 and the catheter 50. The seal may also inhibit the longitudinal passage of microbes along the annular space.

In some embodiments, the isolator 100 may be configured to attach to the patient 10. For example, the isolator 100 may include a flange 140 extending radially outward from the tubular wall 120 defining a feature of the isolator 100 suitable for attachment to the patient 10. For example, the clinician may tape the isolator 100 to the patient 10 via the flange 140.

FIG. 1B is a front cross-sectional view of the isolator 100. As shown, the isolator 100 is generally formed of a tubular shape having a lumen 11 defined by tubular wall 120 extending between a proximal end 101 and a distal end 102. An opening 110 at the proximal end 101 of the isolator 100 is configured to receive the catheter 50 therethrough. Although not required, the opening 110 may include an internal taper 121 to help guide or otherwise facilitate insertion of the catheter 50 through the lumen 111. An external taper 122 at the distal end 102 may facilitate insertion of the isolator along the pathway 12 and may also provide a tip configuration that inhibits abrasion, puncture, or other trauma to the vascular wall.

The tubular wall 120 may be substantially thin and sufficiently flexible so as to conform to the shape (e.g., a curve) of the catheter 50. In some, embodiments, the tubular wall 120 may be sufficiently flexible to allow the body tissue such as the skin layer 11, internal body tissue, or the vascular wall to flatten the lumen 111 when the catheter 50 is not disposed within the isolator 100. The flattening of the lumen 111 may reduce body fluid egress such as bleeding through the lumen 111.

In some embodiments, the isolator 100 may be stretchable. More specifically, the tubular wall 120 may be stretchable in a lateral direction so that a diameter of the isolator 100 may expand to account for or facilitate an interference fit with the catheter 50. In some, embodiments, the isolator 100 may define an interference fit with catheter 50 and interference fit may cause a friction force between the tubular wall 120 and the catheter 50 to inhibit longitudinal displacement of the catheter 50 with respect to the isolator 100. The isolator 100 may be formed any suitable medical grade material including silicone, polyethylene, polypropylene, polytetrafluoroethylene, and the like.

A length 127 of the isolator 100 may be sized to extend at least through the skin layer 11 and/or in some embodiments, extend between the skin surface 11A and the blood vessel 15. As such, the length 127 may be between about 0.2 cm and 1.0 cm, between about 0.3 and 0.7 cm, or about 0.5 cm.

As stated above, the isolator 100 includes antimicrobial properties. For example, in the illustrated embodiment, the isolator 100 may include an antimicrobial coating 130 disposed on any or all surfaces of the isolator 100 including an internal lumen surface. The antimicrobial coating 130 may include chlorhexidine, rifampin, silver sulfadiazine, or any other suitable antimicrobial agents. Alternatively or in addition to surface coating, the antimicrobial coating 130 may be impregnated or otherwise integrated into the isolator material.

As stated above, the isolator 100 may be configured to form a seal with the catheter 50. In some embodiments, the isolator 100 may be sized to define an interference fit with the catheter along an at least a portion of the length of the isolator 100. In some embodiments, although not required, the isolator 100 may include a sealing member 123 to define the seal. For example, the sealing member 123 may be inward annular protrusion disposed on an inside surface of the tubular wall 120. The sealing member 123 may take any form, such as the simple protruding rib as illustrated or some other form, such as a deflectable lip, for example.

The flange 140 discussed above, may facilitate attachment of the isolator 100 to the patient 10 or more specifically to the skin layer 11. The flange 140 may also ensure that the proximal end 101 of the isolator 100 remains external the patient 10. In some embodiments, the flange 140 may be formed of an annular ring extending around a circumference of the isolator 100. The flange 140 may also a plurality of the protrusions extending radially outward of the tubular wall 120.

In some embodiments, the flange 140 may define a plane 140A. The flange 140 (i.e., the plane 140A) may be oriented at an angle 126 with respect to the tubular wall 120 as shown or with respect to a longitudinal axis (not shown) of the isolator 100. In the illustrated embodiment of FIG. 1B, the angle 126 is 90 degrees. In other embodiments, the angle 126 may be an angle other than 90 degrees to accommodate an orientation of the isolator 100 with respect to the skin surface 11A. In other words, the angle 126 may be defined to account for an insertion angle of the isolator 100 with respect to the skin layer 11 as illustrated in FIG. 1A. In some embodiments, the flange 140 may be deflectable with respect to the tubular wall 120 to account for the insertion angle. In some embodiments, the flange 140 may include an adhesive 141 disposed on an underside of the flange 140 to facilitate attachment of the isolator 100 to the skin surface 11A.

In some embodiments, although not required, the isolator 100 may be configured to couple with the catheter 50 so as to inhibit longitudinal displacement of the catheter 50 with respect to the isolator 100. In some embodiments, the interference fit (described above) may inhibit longitudinal and/or rotational displacement of the catheter 50 with respect to the isolator 100. In other embodiments, the isolator 100 may optionally include one or more attachment features 145 to attach the isolator to the catheter 50. The attachment features 145 may be disposed adjacent the proximal end 101 so as to engage corresponding attachment features (not shown) of the catheter 50. As may be appreciated by one of ordinary skill, the attachment features 145 may take any form suitable for inhibit longitudinal and/or rotational displacement of the catheter 50 with respect to the isolator 100.

One method of the using the isolator may generally include inserting the isolator into a defined pathway through the skin layer so that a proximal end of the isolator is disposed outside the patient. In some embodiments, inserting the isolator into a defined pathway may include placing the distal end of the isolator within a blood vessel. The method further generally includes inserting a catheter through the isolator. In some embodiments, the isolator is inserted into the patient prior to inserting the catheter through the isolator, and in other embodiments, the catheter is inserted into the isolator prior to inserting the isolator into the patient. The method may further include manually applying a distally oriented force to the catheter while inserting the isolator into the patient where the catheter transfers the distally oriented force to the isolator. The method may further include inserting the catheter into the isolator so that the distal end of the catheter is proximal the distal end of the isolator while the isolator is disposed outside the patient and thereafter, further inserting the catheter into the isolator so that the distal end of the catheter is distal the distal end of the isolator after the isolator is inserted into the patient. In some embodiments, the method may include attaching the isolator to the catheter to prevent movement of the catheter with respect to the isolator. The method may also include attaching the isolator to the patient to prevent movement of the isolator during a medical procedure.

In some embodiments, the method includes inserting a catheter introducer (or dilator) into the isolator. The method may further include manually applying a distally oriented force to the catheter introducer while inserting the isolator into the patient where the catheter introducer transfers the distally oriented force to the isolator. The method may also include removing the introducer from the isolator. In some embodiments, the method may include threading the isolator onto a guidewire (not show).

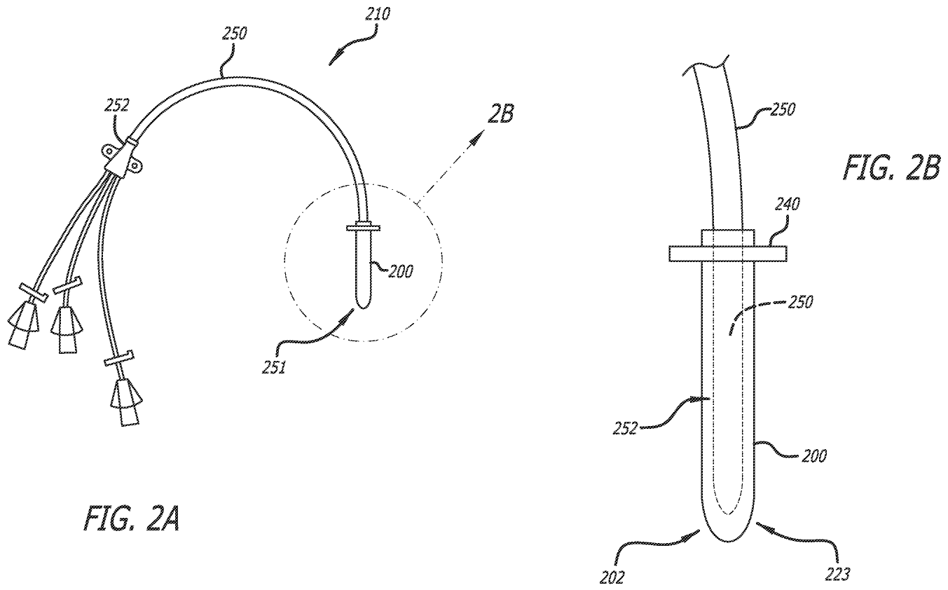

FIGS. 2A-2B illustrate an embodiment of an catheter assembly 210 including a catheter 250 and an isolator 200. The isolator 200 can, in certain respects, resemble components of the isolator 100 described in connection with FIGS. 1A-1B. It will be appreciated that all the illustrated embodiments may have analogous features. Accordingly, like features are designated with like reference numerals, with the leading digits incremented to “2.” For instance, the flange is designated as “140” in FIGS. 1A-1B, and an analogous flange is designated as “240” in FIGS. 2A-2B. Relevant disclosure set forth above regarding similarly identified features thus may not be repeated hereafter. Moreover, specific features of the isolator 100 and related components shown in FIGS. 1A-1B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the isolator of FIGS. 2A-2B. Any suitable combination of the features, and variations of the same, described with respect to the isolator 100 and components illustrated in FIGS. 1A-1B can be employed with the isolator and components of FIGS. 2A-2B, and vice versa. This pattern of disclosure applies equally to further embodiments depicted in subsequent figures and described hereafter.

As shown in FIG. 2A, according to one embodiment, the isolator 200 is coupled with the catheter 250 at a distal end 251 of the catheter 250. In other embodiments, the isolator 200 may be coupled with the catheter 250 at a location other than the distal end 251, such as between the distal end 152 and the hub 252.

FIG. 2B is a detailed illustration of a distal portion of the catheter assembly 210. As shown, a distal tip 252 of the catheter 250 is disposed within the isolator 200 so that the isolator covers the distal tip 252. By covering the distal tip 252, the isolator 200 prevents contact of the distal tip 252 with external objects including the skin surface 11A prior to insertion thus preventing contamination of the distal tip 252.

In some embodiments, the isolator 200 may include a tapered portion 223 to extend over the distal most portion of the distal tip 252. The tapered portion 223 may also help guide the isolator 200 along the pathway 12.

In some embodiments, the isolator 200 may be coupled with the catheter 250 during manufacture of the catheter 250. As such, a method of manufacture of the catheter assembly 210 may include coupling the isolator 200 with the catheter 250 and sterilizing the isolator 200 and the catheter 250 after assembly. In other embodiments, the clinician may couple the isolator 200 with the catheter 250 prior to use.

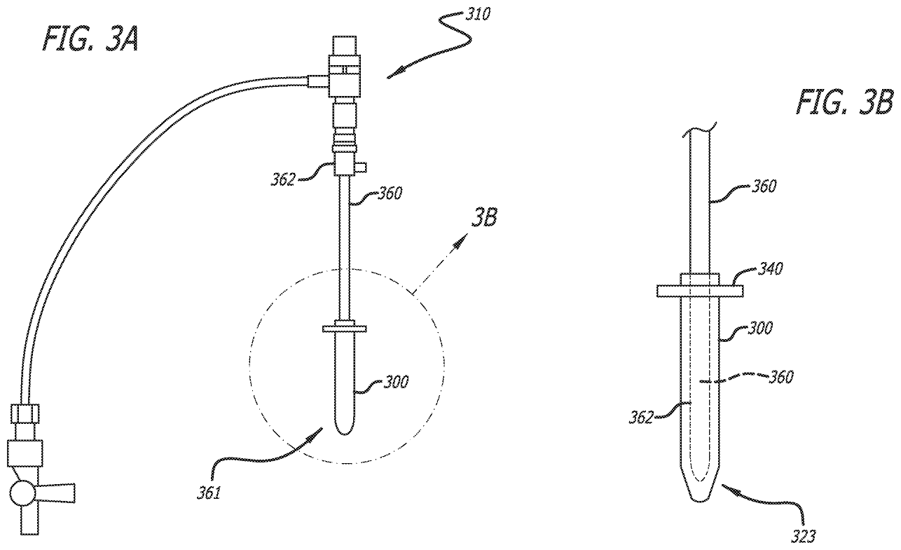

FIGS. 3A-3B illustrate an embodiment of an introducer assembly 310 including an introducer 360 and an isolator 300. As shown in FIG. 3A, according to one embodiment, the isolator 300 is coupled with the introducer 360 at a distal end 361 of the introducer 360. In other embodiments, the isolator 300 may be coupled with the introducer 360 at a location other than the distal end 361, such as between the distal end 361 and the hub 362.

FIG. 3B is a detailed illustration of a distal portion of the introducer assembly 310. As shown, a distal tip 362 of the introducer 360 is disposed within the isolator 300 so that the isolator 300 covers the distal tip 362. By covering the distal tip 362, the isolator 300 prevents contact of the distal tip 362 with external objects including the skin surface 11A prior to insertion thus preventing contamination of the distal tip 362.

In some embodiments, the isolator 300 may include a tapered portion 323 to extend over the distal most portion of the distal tip 362. The tapered portion 323 may also help guide the isolator 300 along the pathway 12.

In some embodiments, the isolator 300 may be coupled with the introducer 360 during manufacture of the introducer 360. As such, a method of manufacture of the introducer assembly 310 may include coupling the isolator 300 with the introducer 360 and sterilizing the isolator 300 along with the introducer 360 after assembly. In other embodiments, the clinician may couple the isolator 300 with the introducer 360 prior to use.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An antimicrobial medical device, comprising:
a tubular body defining a lumen extending between a proximal end and a distal end, the lumen configured to receive a catheter therethrough during an intravascular procedure; and
an antimicrobial material coupled with the tubular body, wherein the tubular body is configured for insertion into a vascular access pathway extending through a skin layer of a patient so that, in use, a wall of the tubular body defines a microbial barrier between the skin layer and the catheter.

2. The antimicrobial medical device of claim 1, wherein the antimicrobial material is a coating applied to the tubular body.

3. The antimicrobial medical device of claim 1, wherein the antimicrobial material is impregnated into a body material of the tubular body.

4. The antimicrobial medical device of claim 1, wherein the tubular body includes a flange protruding radially away from the tubular body, the flange configured to extend along a surface of the skin.

5. The antimicrobial medical device of claim 4, wherein the flange is configured for attachment to the surface of the skin.

6. The antimicrobial medical device of claim 4, wherein the flange includes an adhesive applied to an underside thereof.

7. The antimicrobial medical device of claim 4, wherein the flange is oriented to form an angle with respect to a longitudinal axis of the tubular body.

8. The antimicrobial medical device of claim 1, wherein the tubular body is configured to form a seal with the catheter to prevent egress of body fluid between the tubular body and the catheter.

9. The antimicrobial medical device of claim 8, wherein the tubular body includes an inward annular protrusion configured to form the seal.

10. The antimicrobial medical device of claim 1, wherein the tubular body is sized so that, in use, the distal end of the tubular body is disposed within a blood vessel.

11. The antimicrobial medical device of claim 1, wherein the catheter is a central line catheter.

12. An intravascular catheter assembly, comprising:
a catheter; and
the antimicrobial medical device of claim 1 coupled with the catheter so that the antimicrobial medical device annularly covers at least a portion of the catheter.

13. The intravascular catheter assembly of claim 12, wherein the antimicrobial medical device covers a distal tip of the catheter.

14. A catheter introducer assembly, comprising:
a catheter introducer; and
the antimicrobial medical device of claim 1 coupled with the catheter introducer so that the antimicrobial medical device annularly covers at least a portion of the catheter introducer.

15. The catheter introducer assembly of claim 14, wherein the antimicrobial medical device covers a distal tip of the catheter introducer.

16. A method of placing a catheter within a patient, comprising:
providing an isolator comprising:
a tubular body defining a lumen extending between a proximal end and a distal end, the lumen configured to receive the catheter therethrough during an intravascular procedure; and
an antimicrobial material coupled with the tubular body;
inserting the isolator into a vascular access pathway of the patient;
inserting the catheter through the lumen of the isolator; and
advancing the catheter along a blood vessel of the patient.

17. The method of claim 16, wherein inserting the catheter through the lumen of the isolator forms a fluid seal between the catheter and the isolator.

18. The method of claim 16, wherein the isolator defines a microbial barrier between a skin layer of the patient and the catheter.

19. The method of claim 16, further comprising inserting the isolator into the vascular access pathway so that the distal end of the tubular body is disposed within the blood vessel.

20. The method of claim 16, further comprising inserting the catheter through the lumen of the isolator after inserting the isolator into the vascular access pathway.

21. The method of claim 16, further comprising inserting the isolator into the vascular access pathway so that a flange of the isolator is disposed adjacent a skin surface of the patient.

22. The method of claim 21, further comprising attaching the flange of the isolator to the skin surface.

23. The method of claim 16, further comprising attaching the isolator to the catheter.

24. The method of claim 16, further comprising inserting the catheter into the lumen of the isolator so that a distal end of the catheter is disposed proximal the distal end of the tubular body prior to inserting the isolator into the vascular access pathway.

25. The method of claim 24, wherein inserting the isolator into the vascular access pathway comprises manually applying a distally oriented force to the catheter, wherein the distally oriented force is transferred to the isolator via the catheter.

26. The method of claim 16, further comprising inserting a catheter introducer into the isolator.

27. The method of claim 26, further comprising inserting the catheter introducer into the vascular access pathway after inserting the catheter introducer into the isolator.

28. The method of claim 16, wherein the catheter is a central line catheter.
